(19) United States
(12) Patent Application Publication
(10) Pub. No.: US 2015/0148839 A1
(43) Pub. Date: May 28, 2015

(54) MINIMALLY INVASIVE SURGERY TOOL HAVING VARIABLE BEND

(71) Applicant: Chang Wook Jeong, Seoul (KR)

(72) Inventor: Chang Wook Jeong, Seoul (KR)

(21) Appl. No.: 14/399,887

(22) PCT Filed: May 7, 2013
(86) PCT No.: PCT/KR2013/003976

§ 371 Date: Nov. 7, 2014

(30) Foreign Application Priority Data
May 7, 2012 (KR) 10-2012-0048094

(51) Int. Cl. A61B 17/29

(52) U.S. Cl. CPC A61B 17/29; A61B 2017/00314
USPC 606/208

(57) ABSTRACT

The present invention relates to a minimally invasive surgery tool having a variable bend. According to one embodiment of the present invention, provided is the minimally invasive surgery tool comprising: a shaft; an end constituent element which is connected to one end of the shaft; and a first joint portion which is arranged between the shaft and the end constituent element, wherein the first joint portion comprises a modifiable link portion which can variably form the bend by means of the reaction of at least one wire.

### FIELD OF THE INVENTION

The present invention relates to a minimally invasive surgical instrument having a variable bend.

### BACKGROUND

Minimally invasive surgery is a surgical approach that involves the use of instruments inserted through at least one tiny incision opening to perform a surgery causing minimal tissue trauma in human or animal bodies.

The minimally invasive surgery relatively reduces changes in metabolism of a patient in the period of post-surgical care, so it facilitates rapid recovery of the patient. Therefore, the minimally invasive surgery shortens the length of hospitalization of the patient after the surgery and allows the patient to return to normal physical activities in a short period of time. In addition, the minimally invasive surgery causes less pain and leaves fewer scars on the patient's body after the surgery.

One of the general forms of the minimally invasive surgery is endoscopy. Among the others, a laparoscopy that involves minimally invasive inspection and operation inside abdominal cavity is known as the most general form of endoscopy. To operate a standard laparoscopic surgery, the abdomen of the patient is insufflated with gas and at least one small incision is formed to provide an entrance for laparoscopic surgical instruments, through which a trocar is inserted. When performing the surgery, it is general that a user puts the laparoscopic surgical instruments into a surgical site or the like through the trocar, and manipulates (or controls) the instruments from the outside of abdominal cavity. In general, the laparoscopic surgical instruments include a laparoscope (for observation of a surgical site) and other working tools. Herein, the working tools are similar to the conventional tools used for small incision surgery, except that the end effector or working end of each tool is separated from its handle or the like by a shaft. For instance, the working tools may include a clamp, a grasper, scissors, a stapler, a needle holder, and so forth. Meanwhile, the user monitors the procedure of the surgery through a monitor that displays the images of the surgical site which are taken by the laparoscope. The endoscopic approaches similar to the above are broadly used in retroperitoneoscopy, pelviscopy, arthroscopy, cisternoscopy, sinuscopy, hysteroscopy, nephroscopy, cystoscopy, urethroscopy, pyeloscopy, and so on.

The inventor(s) has developed various minimally invasive surgical instruments useful for the above-mentioned minimally invasive surgeries and has already disclosed the features of the structures and effects of the same in Korean Patent Application Nos. 2008-51248, 2008-61894, 2008-79126 and 2008-90560, the contents of which are to be regarded as being incorporated herein by reference in its entirety. Additionally, the inventor(s) have also introduced a minimally invasive surgical instrument with improved functionality, which is more advantageous for users and patients, in Korean Patent Application Nos. 2010-115152, 2011-3192, 2011-26243 and 2011-29771, the contents of which are to be regarded as being incorporated herein by reference in its entirety.

Herein, the inventor(s) now present a minimally invasive surgical instrument improved from the minimally invasive surgical instruments disclosed in the aforementioned Korean patent applications or other minimally invasive surgical instruments, or a minimally invasive surgical instrument which may be advantageously used together with the above minimally invasive surgical instruments.

### SUMMARY OF THE INVENTION

One object of the present invention is to provide a minimally invasive surgical instrument having a variable bend.

Another object of the invention is to provide a minimally invasive surgical instrument which may have a bend only when necessary and may be easily inserted into or taken out of a human body.

Yet another object of the invention is to allow a surgical observation apparatus to secure a good visual field of operation.

According to one aspect of the invention to achieve the objects as described above, there is provided a minimally invasive surgical instrument comprising a shaft; an end component being connected to one end of the shaft; and a first joint unit being disposed between the shaft and the end component, wherein the first joint unit comprises a modifiable link unit being capable of variably forming a bend by the action of at least one wire.

In addition, there may be further provided other configurations according to the technical idea of the invention.

According to the invention, there is provided a minimally invasive surgical instrument having a variable bend.

According to the invention, there is provided a minimally invasive surgical instrument which may have a bend only when necessary and may be easily inserted into or taken out of a human body.

According to the invention, a surgical observation apparatus may secure a good visual field of operation.

### BRIEF DESCRIPTION OF THE DRAWINGS

FIG. 1 shows a minimally invasive surgical instrument according to one embodiment of the invention.

FIG. 2 shows a case in which an auxiliary joint unit operates to cause the minimally invasive surgical instrument of FIG. 1 to have a bend.

FIG. 3 shows one of wedge-shaped links 402 shown in FIG. 1.

FIG. 4 shows a minimally invasive surgical instrument according to another embodiment of the invention.

FIG. 5 shows a case in which an auxiliary joint unit operates to cause the minimally invasive surgical instrument of FIG. 4 to have a bend.

FIG. 6 shows one of links 402 shown in FIG. 4.

FIG. 7 illustratively shows a case in which a minimally invasive surgical instrument according to the invention is used together with another minimally invasive surgical instrument 700.

FIGS. 8 and 9 are reference views.

### DETAILED DESCRIPTION OF THE PREFERRED EMBODIMENTS

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations or arrangements of individual components within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, preferred embodiments of the present invention will be described in detail with reference to the accompanying drawings to enable those skilled in the art to easily implement the invention.

Meanwhile, it should be understood that the term “connection” herein encompasses a direct connection or an indirect connection (i.e., via separate components) between mechanical or other types of components.

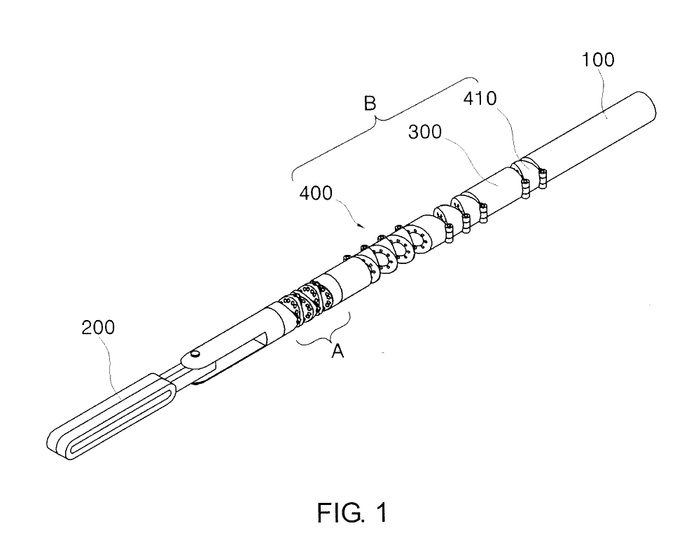

FIG. 1 shows a minimally invasive surgical instrument according to one embodiment of the invention. Reference will be made to FIG. 1.

The minimally invasive surgical instrument may comprise a shaft 100; a surgical end effector 200 being connected to one end of the shaft 100 to use surgical tools (including a surgical observation apparatus such as a laparoscope) or function itself as a surgical tool; a main joint unit A to connect the shaft 100 with the surgical end effector 200 and to allow the surgical end effector 200 to carry out joint motion (the main joint unit A may not be included depending on the type of the minimally invasive surgical instrument); and an auxiliary joint unit B to connect the shaft 100 with the surgical end effector 200 or the main joint unit A and to variably form a bend. (Considering that the separate main joint unit A may be disposed between the auxiliary joint unit B and the surgical end effector 200 in many cases, the auxiliary joint unit B is referred to as such although it may also be referred to as a “variable bend.”) The auxiliary joint unit B may comprise an intermediate link unit 300, a modifiable link unit 400 and an auxiliary link unit 410, as illustrated. The present invention will be discussed herein mainly focusing on the configuration of the auxiliary joint unit B. It should be noted that in connection with the configurations of the shaft 100, the surgical end effector 200, the main joint unit A and the like, those skilled in the art that may refer to the disclosures of the aforementioned Korean patent applications or Korean Patent Application Nos. 2011-86738, 2011-89854, 2011-91515 and 2011-69997 of the inventor(s) (the contents of which are to be regarded as being incorporated herein by reference in its entirety) without limitation, as necessary. Meanwhile, the present invention will be discussed below mainly assuming that the auxiliary joint unit B is disposed between the shaft 100 and the main joint unit A. However, it should be noted that the auxiliary joint unit B may also be disposed between the surgical end effector 200 and the main joint unit A, at the discretion of those skilled in the art who practice this invention.

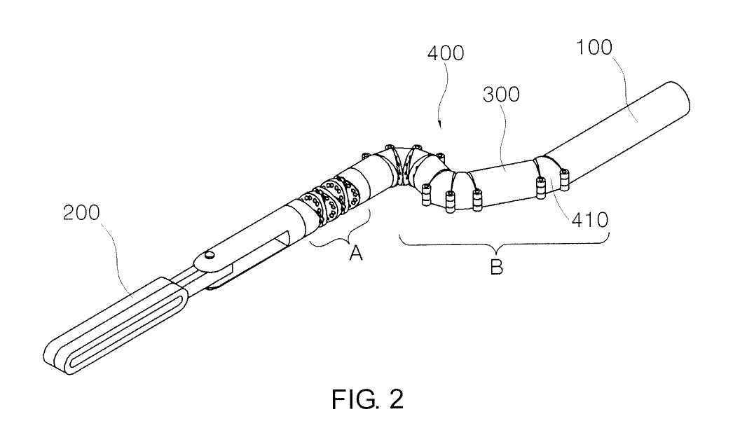

FIG. 2 shows a case in which the auxiliary joint unit operates to cause the minimally invasive surgical instrument of FIG. 1 to have a bend. Reference will also be made to FIG. 2.

The auxiliary joint unit B may allow the minimally invasive surgical instrument to have a bend as shown by the action of wires, when necessary (e.g., when the minimally invasive surgical instrument is used for a surgery as inserted into a human body.) To this end, the auxiliary joint unit B may comprise an intermediate link unit 300 and a modifiable link unit 400 being connected to one side thereof and an auxiliary link unit 410 being connected to the other side thereof.

First, the intermediate link unit 300 is an intermediate part of the auxiliary joint unit 30, and may separate/intermediate between the modifiable link unit 400 and the auxiliary link unit 410 so that the auxiliary joint unit B may form a desirable bend. The intermediate link unit 300 may be omitted in some cases.

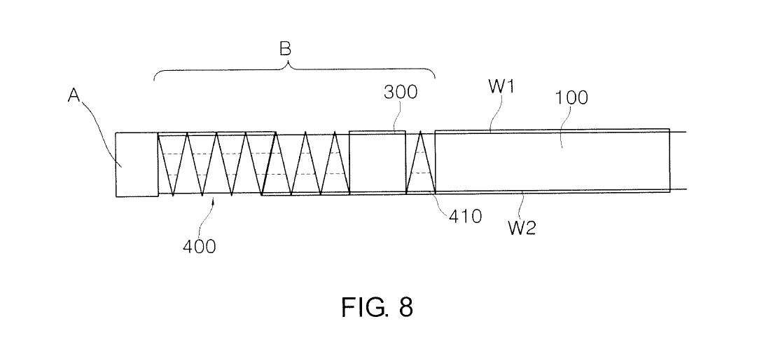

Further, the modifiable link unit 400 may comprise (preferably a plurality of) wedge-shaped links, and may be modified to a bent form when the wires are pulled as bound and fixed to one end of the surgical end effector 200 or the main joint unit A from the shaft 100 through the auxiliary joint unit B consisting of the wedge-shaped links and the like. (The modifiable link unit 400 may return to its original straight form when the wires are not pulled, so that the minimally invasive surgical instrument may be more easily inserted into or taken out of a human body.) In order to form a desirable bend for a surgery, the wires may act within penetration holes disposed opposite to a row of hinges (to be described below) of the wedge-shaped links of at least a part of the modifiable link unit 400. (In connection with the arrangement of the wires W1, W2, further reference may be made to the reference view of FIG. 8.) Meanwhile, as shown in FIGS. 2 and 8, the wedge-shaped links in one part of the modifiable link unit 400 may be configured to face the opposite direction of those in the other part of the modifiable link unit 400.

Meanwhile, the auxiliary link unit 410 may comprise (preferably one or more) wedge-shaped links so as to function like the modifiable link unit 400. The auxiliary link unit 410 may comprise only wedge-shaped links facing one direction so that the auxiliary joint unit B may form a bend only in one direction. The auxiliary link unit 410 may be omitted in some cases.

Meanwhile, it may be preferred that the number of the wedge-shaped links in the one part of the modifiable link unit 400 is equal to that of the wedge-shaped links in the other part of the modifiable link unit 400 plus that of the wedge-shaped links of the auxiliary link unit 410 facing the same direction. In this case, the state of joint motion of the main joint unit A may be advantageously maintained whether a bend is formed in the auxiliary joint unit B or not.

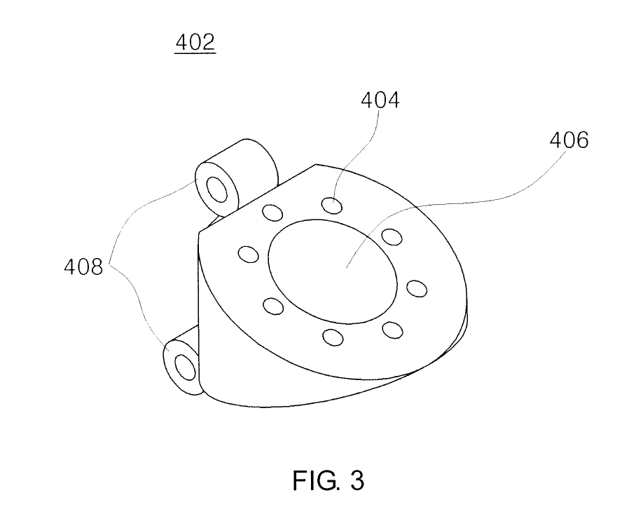

FIG. 3 shows one of wedge-shaped links 402 shown in FIG. 1. Reference will also be made to FIG. 3.

First, the wedge-shaped link 402 may comprise (preferably a plurality of) penetration holes 404 for the above wires which may pass from the shaft 100 to operate the above auxiliary joint unit B. When there exists the main joint unit A, some of the penetration holes 404 may be used for other wires (not shown) which may also pass from the shaft 100 to allow for joint motion of the main joint unit A.

Further, the wedge-shaped link 402 may further comprise a center hole 406 at the center thereof. The center hole 406 may be used as necessary for yet other wires (not shown) or torque transmission members (not shown) which allow for opening/closing operation or roll direction operation of the surgical end effector 200. In connection with components similar to the center hole 406, further reference may be made to the aforementioned Korean patent applications.

In addition, the wedge-shaped link 402 may comprise hinges 408 at one wider side thereof so that it may be connected to other wedge-shaped links or adjacent components.

The shape of the wedge-shaped link 402 is not necessarily limited to the illustrated one. For example, one narrower side of the wedge-shaped link 402 may have a surface structure instead of a sharp structure as shown. It is apparent that at least some of the above structural features of the wedge-shaped link 402 may also be common to other wedge-shaped links. Further, the shape of the link 420 is not necessarily limited to a wedge as long as the technical idea of the invention is achieved.

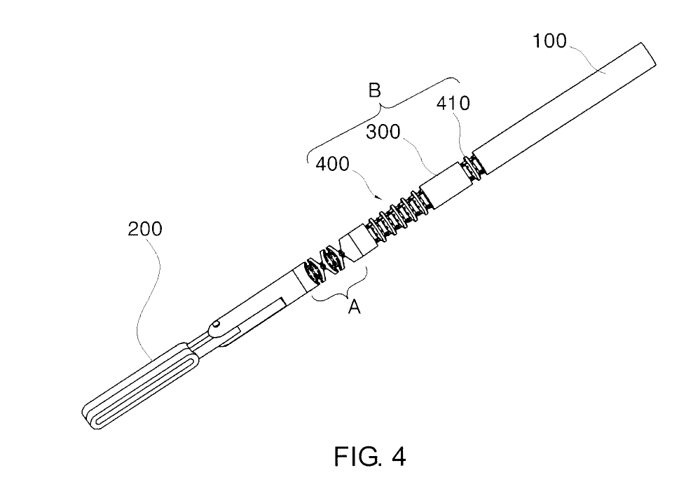

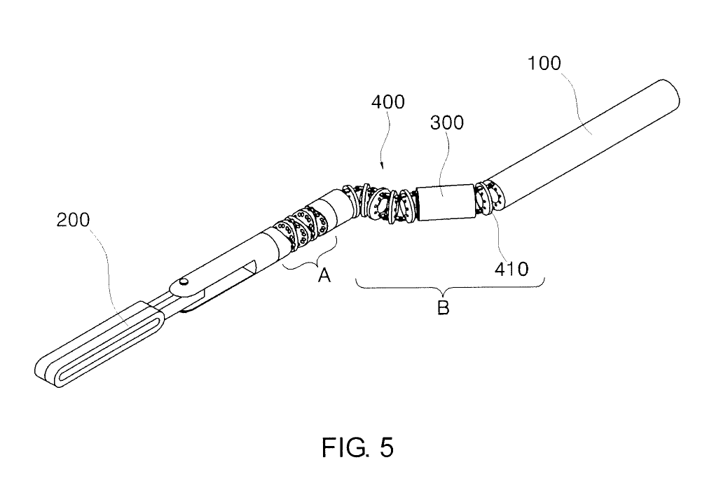

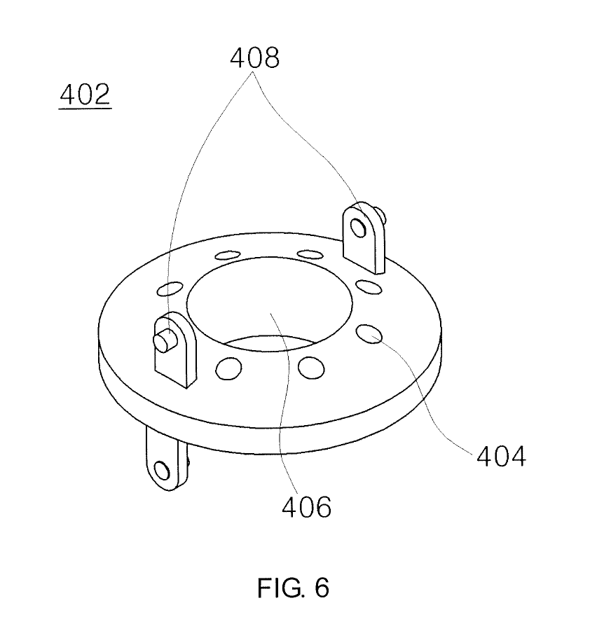

FIG. 4 shows a minimally invasive surgical instrument according to another embodiment of the invention. FIG. 5 shows a case in which an auxiliary joint unit operates to cause the minimally invasive surgical instrument of FIG. 4 to have a bend. Further, FIG. 6 shows one of links 402 shown in FIG. 4.

The basic configuration of the minimally invasive surgical instrument according to this embodiment is similar to that of the minimally invasive surgical instrument according to the foregoing embodiment. However, there are differences in the configuration of the modifiable link unit 400 and/or the auxiliary link unit 410 and that of the wires. Accordingly, the differences in the above configurations will be mainly discussed below. Reference will be made to FIGS. 4 to 6.

In this embodiment, the modifiable link unit 400 and the auxiliary link unit 410 may not be configured with wedge-shaped links but with links having the same or similar shape as that of joint links disclosed in Korean Patent Application Nos. 2011-3192 and 2011-86738. (In connection with the shape or feature of such links, further reference may be made to FIG. 6.) Here, there may be connected a plurality of such links having substantially the same shape and facing substantially the same direction (i.e., so that an alternate arrangement may be avoided).

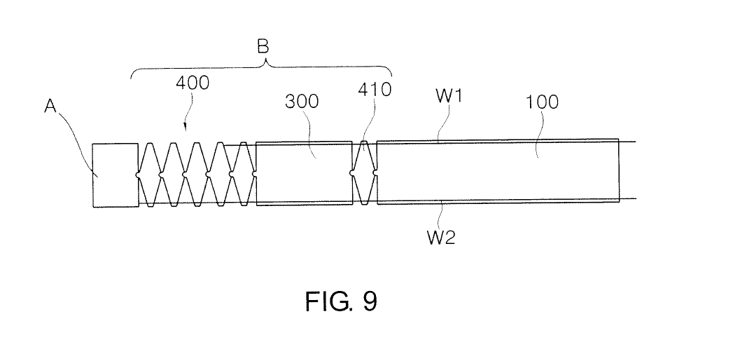

Further, in this embodiment, a first wire may be connected only up to a link in the middle of the modifiable link unit 400, whereas a second wire opposite to the first wire is connected to one end of the surgical end effector 200 or the main joint unit A. (In this case, it may be preferred in the modifiable link unit 400 and the auxiliary link unit 410 that the number of links which the first wire does not reach is equal to that of links which the first wire reaches.) Accordingly, pulling the first wire may cause that the link to which the first wire is bound and fixed and the links closer to the shaft 100 than the above link are narrowed at a corresponding side and spread at the opposite side, and as a reaction thereto, the remaining links in the modifiable link unit 400 operate in the opposite way. Therefore, according to this embodiment, the auxiliary joint unit B may have an appropriate bend by only pulling the first wire. Meanwhile, also in this embodiment, the auxiliary link unit 410 may cause the auxiliary joint unit B to form a bend only in one direction, as shown in FIG. 5. In connection with the above-described arrangement of the first and second wires W1 and W2, further reference may be made to the reference view of FIG. 9.

Meanwhile, the link 402 according to this embodiment may also comprise penetration holes 404, a center hole 406 and hinges 408, similarly to the wedge-shaped link according to the foregoing embodiment.

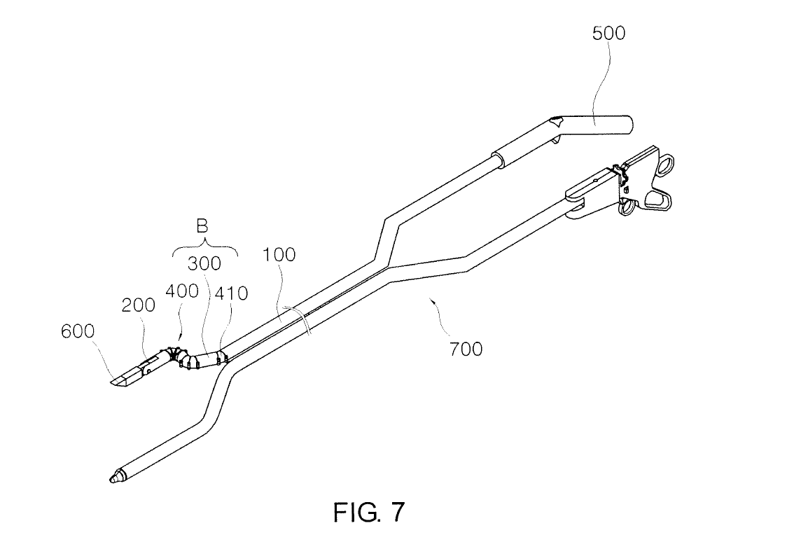

FIG. 7 illustratively shows a case in which a minimally invasive surgical instrument according to the invention is used together with another minimally invasive surgical instrument 700. (Here, the reference numeral 500 indicates a handling unit or the like of the minimally invasive surgical instrument, which may be similar to the handling units disclosed in the aforementioned Korean patent applications, and thus the detailed description thereof will be omitted.)

As shown in FIG. 7, the minimally invasive surgical instrument according to the invention may allow a bend to be formed in an auxiliary joint unit B, thereby avoiding collision with the other minimally invasive surgical instrument 700 within a human body. In many cases, the other minimally invasive surgical instrument 700 may have a bend (see Korean Patent Application Nos. 2011-26243 and 2011-29771 of the inventor(s)), and particularly the minimally invasive surgical instrument according to the invention may be advantageously used together with the other minimally invasive surgical instrument 700 as above.

Further, when a surgical end effector 200 uses or functions itself as a surgical observation apparatus 600 such as a laparoscope as illustrated, forming a bend in the auxiliary joint unit B according to the invention may secure a good visual field of operation, i.e. a visual field which allows observing a surgical field where the other minimally invasive surgical instrument 700 is operated at an appropriate distance and angle in a forward direction.

Although the present invention has been described in terms of specific items such as detailed components as well as the limited embodiments and the drawings, they are only provided to help general understanding of the invention, and the present invention is not limited to the above embodiments. It will be appreciated by those skilled in the art that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A minimally invasive surgical instrument, comprising:
a shaft;
an end component being connected to one end of the shaft; and
a first joint unit being disposed between the shaft and the end component,
wherein the first joint unit comprises a modifiable link unit being capable of variably forming a bend by the action of at least one wire.

2. A minimally invasive surgical instrument as claimed in claim 1, wherein the end component comprises a surgical end effector and a second joint unit.

3. A minimally invasive surgical instrument as claimed in claim 1, wherein the at least one wire is fixed to the end component.

4. A minimally invasive surgical instrument as claimed in claim 1, wherein the modifiable link unit comprises a plurality of wedge-shaped links.

5. A minimally invasive surgical instrument as claimed in claim 4, wherein one part of the plurality of wedge-shaped links and the other part thereof face opposite directions.

6. A minimally invasive surgical instrument as claimed in claim 5, wherein the first joint unit further comprises an intermediate link unit.

7. A minimally invasive surgical instrument as claimed in claim 6, wherein the first joint unit further comprises an auxiliary link unit being separated from the modifiable link unit by the intermediate link unit.

8. A minimally invasive surgical instrument as claimed in claim 7, wherein the auxiliary link unit comprises one or more wedge-shaped links, and the one or more wedge-shaped links face one direction.

9. A minimally invasive surgical instrument as claimed in claim 8, wherein the number of the one part of the plurality of wedge-shaped links is equal to that of the other part of the plurality of wedge-shaped links plus that of the one or more wedge-shaped links of the auxiliary link unit.

10. A minimally invasive surgical instrument as claimed in claim 1, wherein the modifiable link unit comprises a plurality of joint links.

11. A minimally invasive surgical instrument as claimed in claim 10, wherein the at least one wire comprises a first wire and a second wire,
the first wire is fixed to one of the plurality of joint links, and
the second wire is fixed to the end component.
